(19) United States
(12) Patent Application Publication
(10) Pub. No.: US 2025/0248781 A1
(43) Pub. Date: Aug. 7, 2025

(54) ENDOSCOPY TRAY

(71) Applicant: GI Reviewers, LLC, Brookline, MA (US)

(72) Inventors: Daniel Shlomo Mishkin, Brookline, MA (US); Kenneth Edward Wolcott, Fox Point, WI (US); Vito DeMonte, North Babylon, NY (US)

(73) Assignee: GI Reviewers, LLC, Brookline, MA (US)

(21) Appl. No.: 18/435,577

(22) Filed: Feb. 7, 2024

(51) Int. Cl. A61B 50/33; A61B 50/00; A61B 50/30; A61B 50/36; A61M 25/00

(52) U.S. Cl. CPC A61B 50/33; A61B 50/36; A61B 2050/0055; A61B 2050/0084; A61B 2050/3014; A61M 25/002

(57) ABSTRACT

A fluid retaining tray including a tray surface panel having a fluid retaining space. A first side panel extends outwardly from a first side edge of the tray surface panel and rotatable relative thereto between a storage position and an operative position. The first side panel has a first flap rotatably secured thereto. The flap has an engagement element selectively securable to the tray surface panel for securing the first side panel in an operative position. The first side panel has a distal end including a first recess adapted to retain a first tube end above the fluid retaining space.

### FIELD OF TECHNOLOGY

This disclosure relates to a device for retaining spilled or dripped liquids and in particular, a tray for use in an endoscopy procedure for retaining fluids that drip from tubing and fluid ports associated with the procedure.

### BACKGROUND

In an endoscopy procedure, an endoscope is inserted inside a patient's body to take images and examine organs and other structures. There are many types of endoscopy that view different organs. The most common types of endoscopy include colonoscopy, upper endoscopy and cystoscopy.

For a colonoscopy, the endoscope's distal end is inserted into a patient and the proximal end of the endoscope is connected to an endoscopy processor or control center. The processor receives signals from the endoscope's camera and converts them into video images that can be displayed on a monitor. It also controls various functions of the endoscope, such as air and water insufflation, suction, and image enhancement features. Tubes for suction and fluid supply are also connected to the proximal end of the endoscope.

When the procedure is completed, the endoscope is disconnected from the processor and the tubes are disconnected from the endoscope. The current standard of practice is to have a turn over of the equipment, i.e., the used/soiled scope is removed as the room goes from dirty to clean, and a new clean scope is attached. During this turn over process the procedure area will become dirty with dripped fluids and then wiped down afterwards. Specifically, when the tubes are disconnected, fluid tends to drip out of them, which can contaminate surrounding surfaces such as the processor, processor cart or floor. This requires the area to be cleaned before the next procedure is performed. This is time consuming and also not ideal as it requires more bending and many times getting under the cart to clean. All the while, making it more taxing on the person performing this task. This cleaning can be time consuming and difficult to fully complete since the procedure apparatus may need to be moved and relocated. Pads held by technicians while removing the tubes can catch some of the fluid. However, this is not a reliable way to retain the fluid and some still can fall on the floor and surrounding surfaces.

Accordingly, it would be desirable to provide a device for reliably capturing and retaining fluids generated during an endoscopy procedure help keep the area from getting dirty/wet, in order to reduce the time and effort to “turn over” the endoscopy procedure room.

### SUMMARY

The present disclosure provides a fluid retaining tray including a tray surface panel having a fluid retaining space. A first side panel extends outwardly from a first side edge of the tray surface panel and rotatable relative thereto between a storage position and an operative position. The first side panel has a first flap rotatably secured thereto. The flap has an engagement element selectively securable to the tray surface panel for securing the first side panel in an operative position. The first side panel has a distal end including a first recess adapted to retain a first tube end above the fluid retaining space.

The present disclosure also provides an endoscopy tray including a tray surface panel having an upstanding wall extending therefrom. The wall includes a plurality of wall elements joined at their ends to form an enclosed perimeter. The wall elements and tray surface panel defining a fluid retaining space. A first side panel extends from a first side edge of the tray surface panel and rotatable relative thereto between a storage position and an operative position, the first side panel having a first flap rotatably secured thereto. The flap has an engagement element selectively securable to a first wall element of the plurality of wall elements for securing the first side panel in an operative position. The first side panel has a distal end including a first recess adapted to retain a tube end above the fluid retaining space.

The present disclosure further provides a foldable endoscopy tray including a tray surface panel having a fluid retaining space defined between a first and side edge of the tray surface panel. A first side panel extends outwardly from the first side edge of the tray surface panel and a second side panel extending from the second side edge of the tray surface panel. The first and second side panels each are rotatable relative to the tray surface panel between a storage position and an operative position. The first side panel has a first flap rotatably secured thereto and the first flap having an engagement element. The first flap is foldable relative to the first side panel into a position wherein the engagement element engages the tray surface panel to secure the first side panel in an operative position. The first side panel has a distal end including a first recess adapted to retain a first tube end above the fluid retaining space. The tray surface panel has a third side edge disposed between the first and second side edges and a tray retainer extends from the third side edge.

### BRIEF DESCRIPTION OF THE DRAWINGS

FIG. 1 is a perspective view of the tray of the present disclosure positioned adjacent an endoscope processor.

FIG. 2 is a top perspective view of the tray of FIG. 1.

FIG. 3 is a bottom perspective view of the tray.

FIG. 4 is a back elevational view of the tray in the operational position.

FIG. 5 is a back elevational view of the tray in the operational position.

FIG. 6 is a detail view of a tube holding element.

FIG. 7 is detail view a securement arrangement.

FIG. 8 is a top perspective view of an alternative embodiment of the tray.

FIG. 9 is a top perspective view of the tray of FIG. 9 with an adsorbent pad.

FIG. 10 is a perspective view of the tray of FIG. 9.

FIGS. 11 and 12 are detailed views of an alternative embodiment of a securement arrangement.

FIG. 13 is a perspective view of a further alternative embodiment of the tray showing the tray being inserted into a tray receptacle disposed between the endoscopy processor and a support surface.

FIG. 14 is a top perspective view of the tray of FIG. 13.

FIG. 15 is a top perspective view of the tray receptacle.

FIG. 16 is a front view of the tray receptacle.

### DETAILED DESCRIPTION

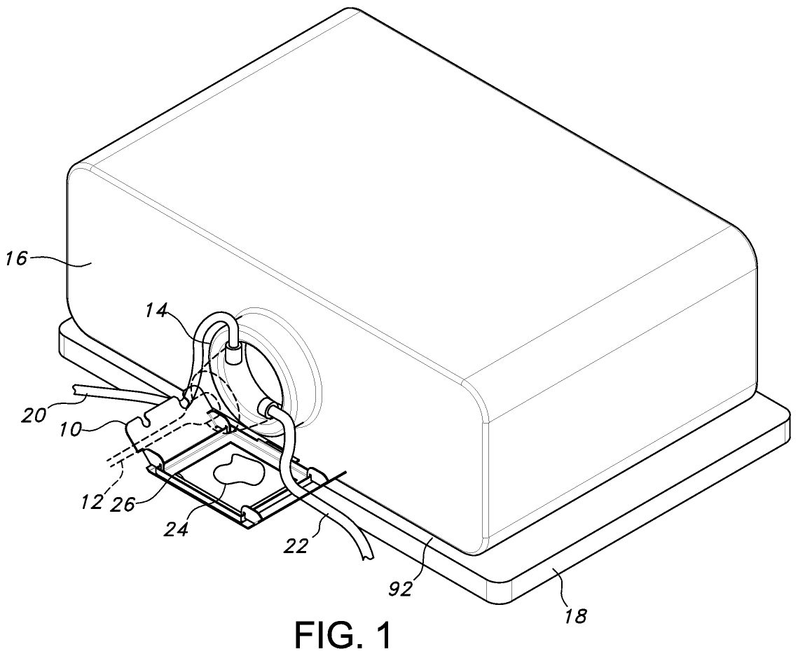

With reference to FIG. 1, a tray 10 for collecting and retaining fluid is shown. The tray 10 may be used during an endoscopy procedure to prevent fluid from inadvertently spilling on the floor or surrounding surfaces. An endoscope 12 has a proximal end 14, which is connected to a processing device 16, or controller placed on a support surface such as a cart 18. The endoscope proximal end 14 has connections for tubing such as suction tube 20 and a water tube 22. When these tubes are disconnected from the endoscope, fluid can drip out from the tubes and corresponding fluid connection ports and fall on the floor or cart. The tray 10 is used to contain such fluids 24 and keep them from contaminating surrounding surfaces.

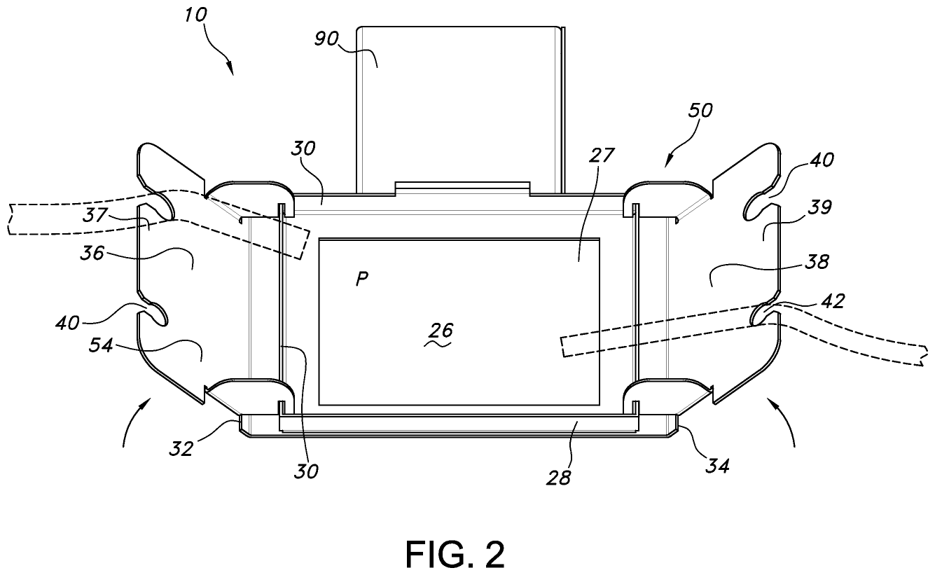

With additional reference to FIGS. 2-4, the tray 10 includes a tray surface panel 26 having a fluid retaining space 27 that is adapted to be positioned below the endoscope proximal end 14 (FIG. 1). In one embodiment, the tray surface panel 26 has an upstanding wall 28 extending therefrom. The wall 28 includes a plurality of wall elements 30 joined at their ends to form an enclosed perimeter. The wall elements 30 and tray surface panel 26 defining the fluid retaining space 27. The wall may be formed separately from the surface panel and be secured thereto. Alternatively, no wall is used and the fluid can be retained by way of an absorbent pad as will be described below.

The tray 10 may be formed of paperboard or cardboard material that has a water resistant coating. Alternatively, the tray 10 may be formed of plastic or metal sheeting or similar planar and foldable material. The tray 10 can thus be formed from foldable blanks. It is also contemplated that the tray could be formed by molding or otherwise methods of forming material as is known in the art.

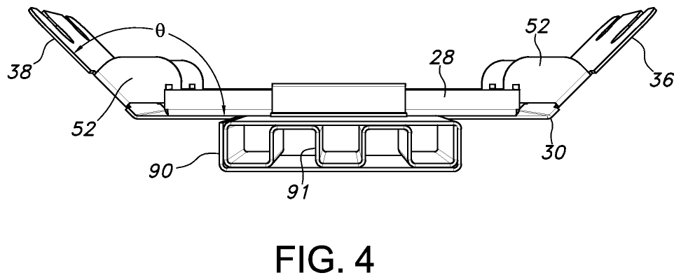

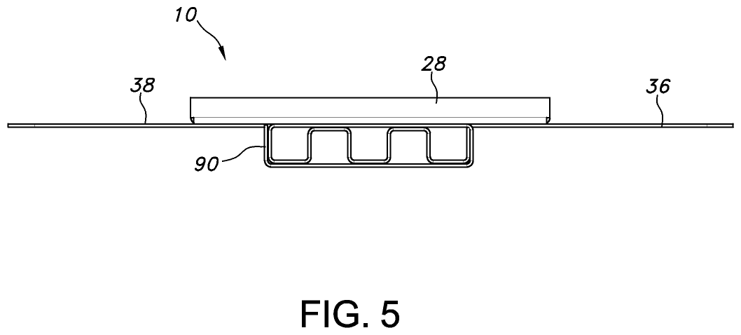

The tray surface panel 26 has opposed first and second sides, 32 and 34 respectively. A first side panel 36 extends from the first side 32 and terminates in a distal end 37. A second side panel 38 extends from the tray surface panel second side 34 and terminates in a distal end 39. The first and second side panels are each rotatable relative to the surface panel 26 along a fold line between a storage position and an operative position. In the storage position, shown in FIG. 5, the first and second side panels 36 and 38 lie in the same plane as the tray surface panel 26 such that the tray 10 is essentially flat. This position is beneficial for transportation and storage since the trays can be staked on top of each other. In the operative position, shown in FIGS. 1 and 4, the first and second side panels 36 and 38 are folded along a respective fold line relative to the tray surface panel such that side panels distal ends 37 and 39 are positioned above the plane P in which the tray panel surface lies. In one embodiment, the first and second side panels 36 and 38 form an angle Q with the surface panel generally in the range of between 90 and 150 degrees relative to the tray surface panel 26. A range of 110 to 130 degrees is preferable. These ranges are intended to be exemplary and not limiting.

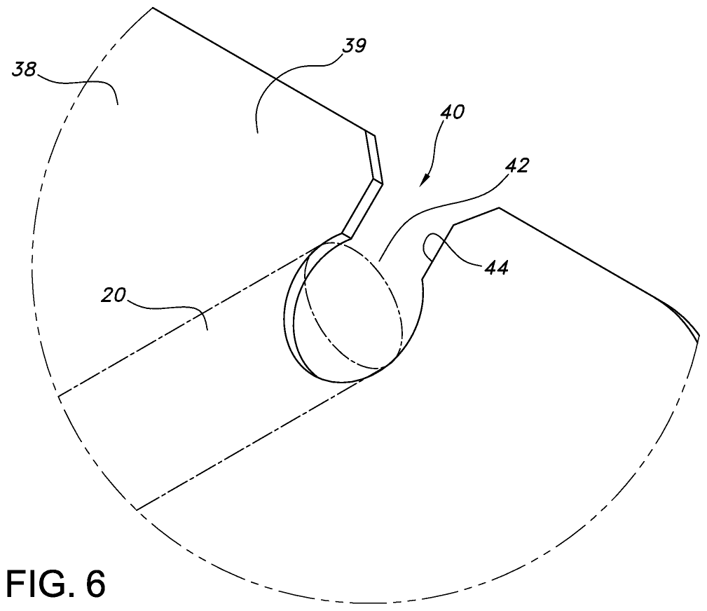

With reference to FIGS. 2 and 6, the first and second side panel distal ends 37 and 39 each include one or more tube holding structures 40. This holding structure 40 is configured to support and hold a tube 20, 22 used in an endoscopy procedure, such that the tube end lies above and preferably over the tray surface panel 26. The tube holding structure 40 may be in the form of one or more recesses 42 formed in the distal end of the side panels. The recesses 42 may have a reduced width portion or neck 44 in a medial portion of the recess. A tube is inserted into the recess and pressed past the neck into the bottom of the recess. The flexibility of the tubing 20, 22 allows it to deform to pass through the neck and be retained on the panel. The tubing 20, 22 can be slid relative to the side panel such that the end of the tube is positioned over the tray panel surface 26. The retained position of the tubing 20, 22 also positions the tubing end above the fluid level in the tube which helps prevent fluid from dripping out of the tube.

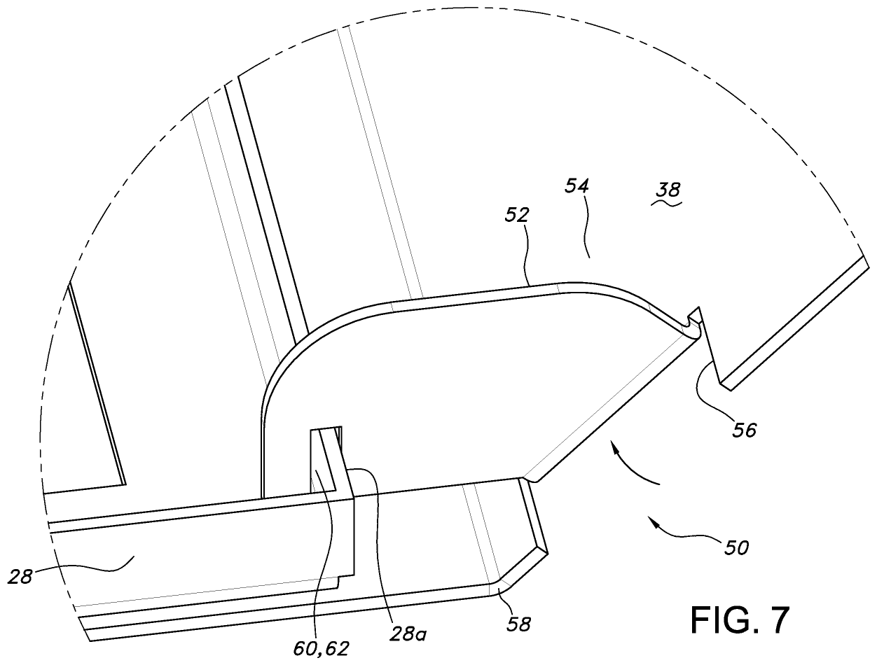

With additional reference to FIG. 7, the first and second side panels 36 and 38 are retained in the operative position by a securement arrangement 50. The securement arrangement 50 includes one or more flaps 52 rotatably secured to a main body portion 54 of the first and second side panels. The first and second side panels, 36 and 38, and the flaps 52 may be formed from the same single piece of material. Cut lines 56 formed in the first and second side panels define the edges of the flaps 52 and the flaps are foldable along a fold line 58 relative to the main body portion of the side panels. The flaps 52 each have an engagement element 60 selectively securable to the tray surface panel 26 for securing the side panels in an operative position. In one embodiment shown in FIG. 7, the engagement element 60 includes a notch 62 configured to receive therein a portion of the perimeter wall 28a to selectively secure the side panel in the operative position. A user rotates the flaps 52 away from the side panel main body portion 54, and manipulates the flaps until a portion of wall 28 enters the notches 62. The inherent flexibility of the relatively thin material from which the flaps are made helps the flaps 52 to be manipulated such that they are secured to the wall 28. In this embodiment, the flaps 52 hook onto the wall 28 and retain the first and second side panel 36 and 38 in the operative position. In order to return the side panels to the storage position, the flaps can be rotated such that the wall moves out of the notch 62.

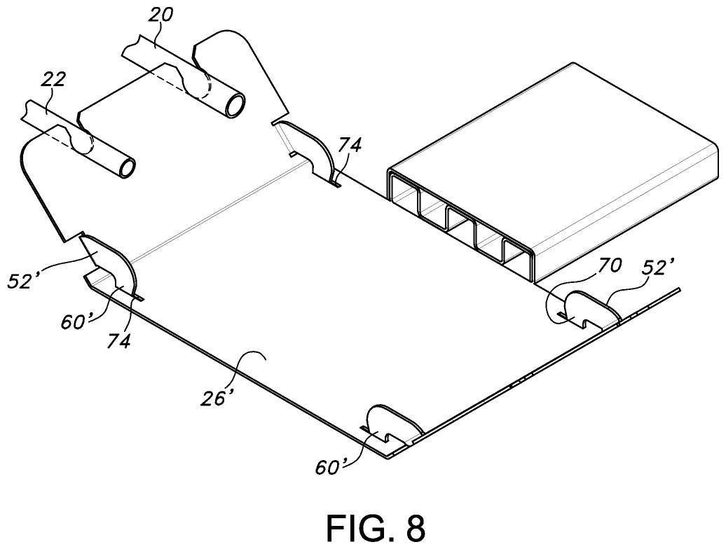

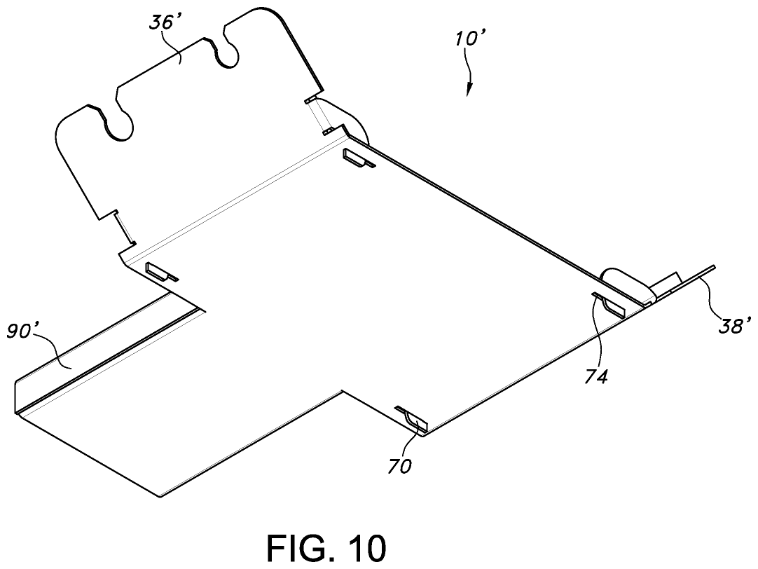

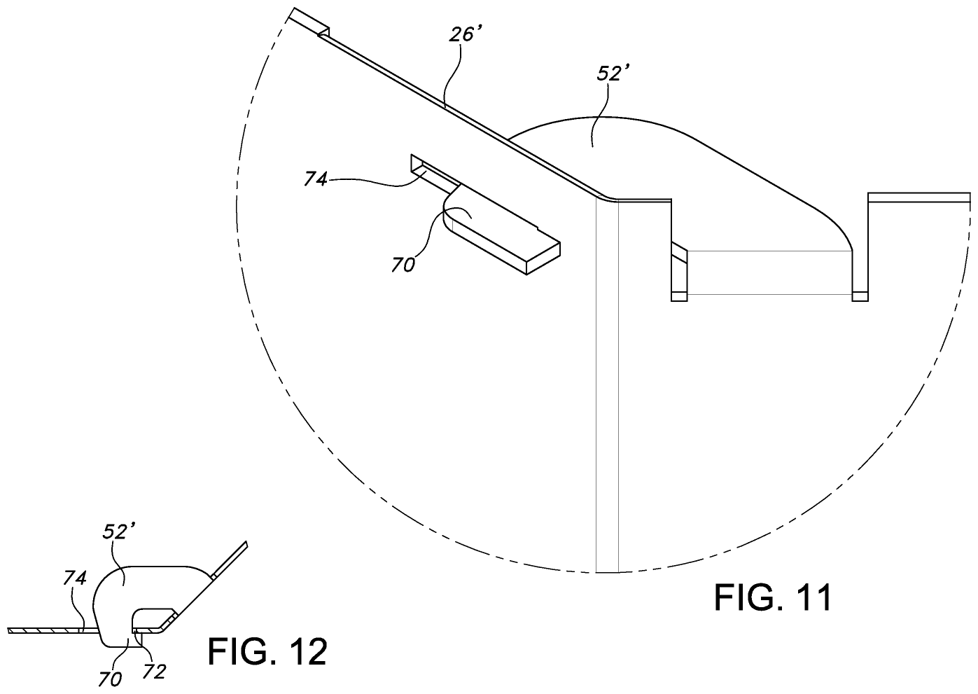

An alterative embodiment of the tray is shown in FIGS. 8-12. Tray 10′ is similar to the tray 10″ described above but tray 10′ includes a tray surface panel 26′ that is a flat surface having no upstanding wall. In order to accommodate the wall-less design, the engagement element 60′ is modified to include a tab 70 extending from the distal end of the flaps 52′. The tabs 70 each includes a notch 72 (FIG. 12) therein. In order to secure the first and second side panels 36′ and 38′ in the operative position, the flaps 52′ are rotated such that they are generally perpendicular to the side panel central portion. The side panel is rotated toward the operable position and the tab is inserted in a corresponding slot 74 formed in the tray surface panel 26′. The slots are defined by edges. Each flap 52′ may include a tab 70 and the tray surface panel includes a corresponding number of slots 74 to accommodate each of the tabs. The side panels 36′ and 38′ have a tendency to return to the initial position and this causes the tab notch 72 to receive the edge of the slot therein, thus retaining the side panels 36′ and 38′ in the operative position.

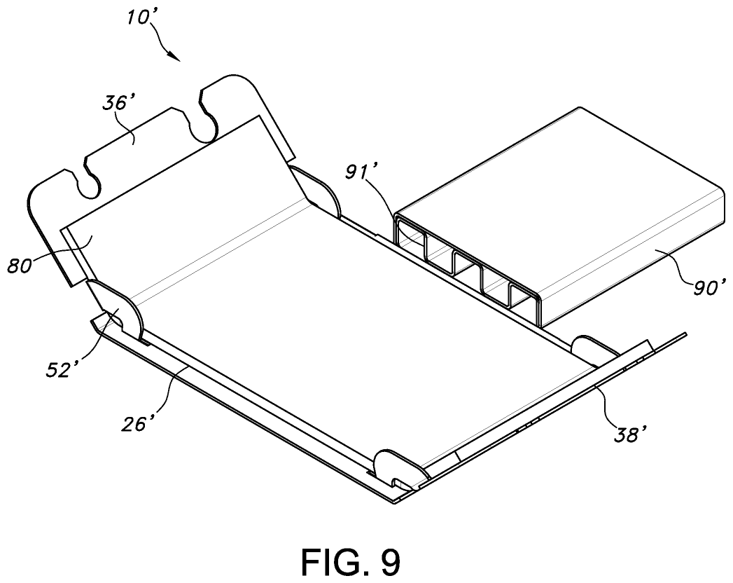

In either of the above-described embodiments, a pad of absorbent material 80 may be secured to the tray surface panel 26, 26′ to help retain dripped fluid. As shown in FIG. 9, the pad 80 may extend up onto the side panels to receive any fluid which falls from the tubing 20, 22. The pad 80 may be one continuous piece of material or could be formed of separate pieces. For example, in the embodiment with the perimeter wall 28, one piece of pad material may cover the surface within the perimeter and other pieces may be used to cover the two side panels 36 and 38. In the embodiment without a wall, one continuous piece of material may be used. The pad, or pads, may be removable from the tray to permit the soiled pads to be removed and discarded but the tray reused. Alternatively, the pad(s) 80 could be fixedly secured to the tray and, when soiled, the entire tray is discarded.

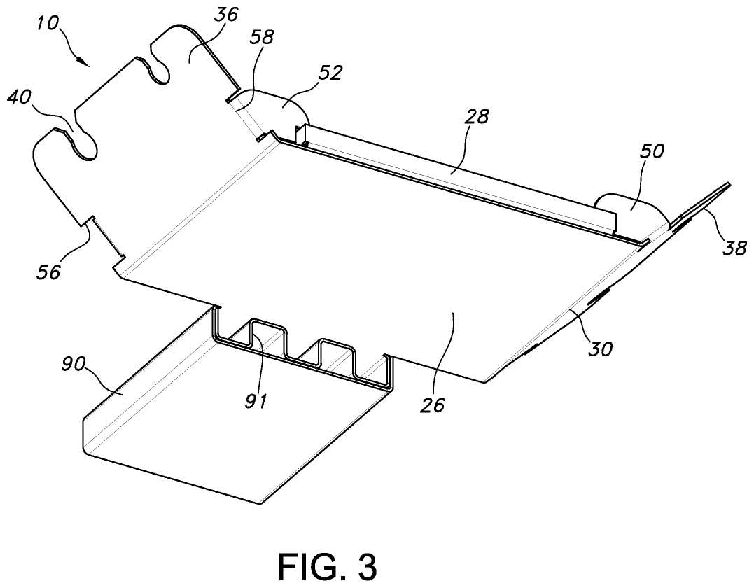

During an endoscopy procedure, the tray 10 is preferably retained below the endoscope connection location. In order to affect this placement, the tray 10 has a tray retainer 90 extending outwardly from a third side of the tray surface panel disposed between the first and second side. The tray retainer 90 is configured to sit between a space 92 formed between the processor 16 and the support surface 18 on which it sits. In one embodiment shown in FIGS. 3 and 4, the retainer 90 has first and second opposed surfaces separated by support ribs, and may have a generally rectangular box-like cross-section. The retainer may be formed from the same piece of material used to form the tray surface panel 26. Alternatively, the retainer could be formed separately therefrom and secured to it by an adhesive or other fastening-type element. In one embodiment, the retainer extends above the tray surface panel 26, as shown in FIG. 8, and in an alternative embodiment is resends below the tray surface panel 26, as shown in FIG. 3.

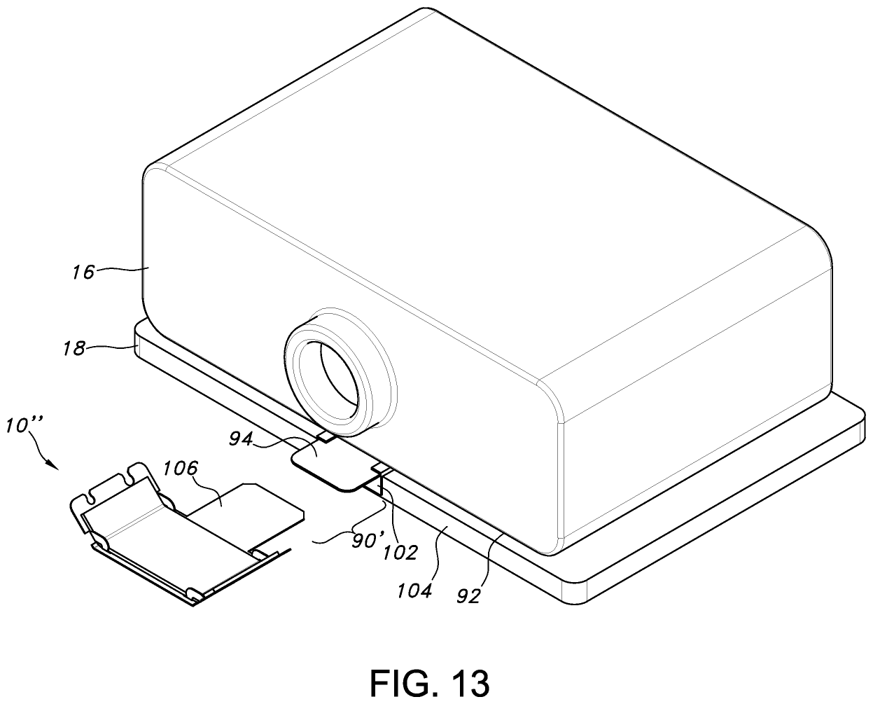

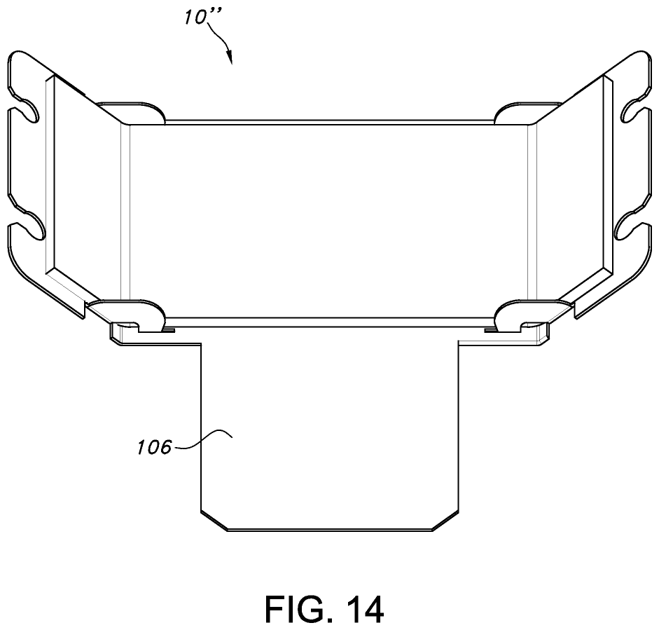

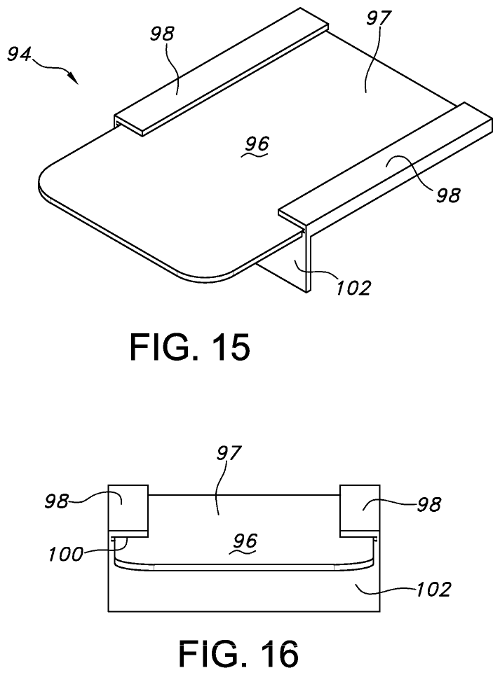

A further alternative embodiment of the tray is shown in FIGS. 13-16, Tray 10′″ is similar to tray 10″ described above, but the tray retainer 90′ is a two-element component. One element is a tray receptacle 94 configured to be inserted between the processor 16 and the support surface 18. The tray receptacle 94 is separate from the tray surface panel. The second component of the tray retainer 90′ is in a flat tongue-like element 106 extending from the tray surface panel. The tray receptacle 94 includes a planar panel 96 having a top surface 97 bounded along opposed side edges by in-turned portions 98 forming longitudinally extending slots 100. A stop 102 extends from the bottom surface of the planar panel 104. The stop 102 prevents the receptacle from being inserted too far between the controller and support surface. The bottom surface 104 may include an attachment device (not shown) such as an adhesive or hook and loop material to fix the position of the receptacle relative to the processor 16 and support surface 18. The second component of the tray retainer is in a flat tongue-like element 106 extending outwardly from the tray surface panel 26. This is a relatively thin element formed integrally with the tray surface panel, and is an extension thereof. The tongue 106 is adapted to be slid into the opposed slots 100. Accordingly, when a user wants to replace the tray 10, they can slide the existing tray out of the receptacle 94 and slide in a new tray 10. The tray receptacle 94 may be formed of the same type of material as the tray or may be formed of a different material. For example, the tray could be formed of a paperboard material and the tray receptacle 94 could be formed of plastic. While the two piece tray retainer 90′ is shown with the tray having a wall-less tray surface panel, it is contemplated that this two piece design tray retainer 90′ could also be incorporated into the tray 10 having the perimeter wall.

Although specific example embodiments have been described, it will be evident that various modifications and changes are made to these embodiments without departing from the broader scope of the inventive subject matter described herein. Accordingly, the specification and drawings are to be regarded in an illustrative rather than a restrictive sense. The accompanying drawings that form a part hereof, show by way of illustration, and without limitation, specific embodiments in which the subject matter are practiced. The embodiments illustrated are described in sufficient detail to enable those skilled in the art to practice the teachings herein. Other embodiments are utilized and derived therefrom, such that structural and logical substitutions and changes are made without departing from the scope of this disclosure. This Detailed Description, therefore, is not to be taken in a limiting sense, and the scope of various embodiments is defined only by the appended claims, along with the full range of equivalents to which such claims are entitled.

What is claimed is:

1. A fluid retaining tray comprising:
a tray surface panel having a fluid retaining space; and
a first side panel extending outwardly from a first side edge of the tray surface panel and rotatable relative thereto between a storage position and an operative position, the first side panel having a first flap rotatably secured thereto, the flap having an engagement element selectively securable to the tray surface panel for securing the first side panel in an operative position, the first side panel having a distal end including a first recess adapted to retain a first tube end above the fluid retaining space.

2. The tray as defined in claim 1, including a second side panel extending from a second side edge of the tray surface panel, the second side panel being rotatable relative to the tray surface panel between a storage position and an operative position, the second side panel having a first flap rotatably secured thereto, the flap having an engagement element selectively securable to the tray surface panel for securing the second side panel in an operative position, the second side panel having a distal end including a first recess adapted to retain a second tube end above the fluid retaining space.

3. The tray as defined in claim 1, wherein the first flap includes a tab insertable in a first slot formed in the tray surface panel, wherein the first tab cooperates with the first slot to retain the first side panel in the operable position.

4. The tray as defined in claim 1, wherein the tray surface panel includes an upstanding wall extending therefrom, the wall including a plurality of wall elements joined at their ends to form an enclosed perimeter, the wall elements and tray surface panel defining the fluid retaining space.

5. The tray as defined in claim 4, wherein the flap has an engagement element that is selectively securable to the wall.

6. The tray as defined in claim 4, wherein the flap engagement element defines a notch and the notch is configured to receive a portion of the wall to retain the first flap in the operating position.

7. The tray as defined in claim 1, wherein the first side panel forms an angle with the tray surface panel in the range of 90 to 150 degrees.

8. The tray as defined in claim 1, wherein an absorbent pad is disposed on the tray surface panel.

9. The tray as defined in claim 8, wherein the tray surface panel has a third side edge disposed between the first and second side edges and the tray retainer extends from the third side edge.

10. The tray as defined in claim 9, wherein the tray retainer includes a first element extending from the tray surface panel.

11. The tray as defined in claim 10, wherein the tray retainer includes a tray receptacle for receiving therein the first element.

12. The tray as defined in claim 10, wherein the first element is a tongue-like element having a planar surface and the receptacle having a pair of opposed side edges each defining a slot, the first element being slidably received in the slots.

13. The tray as defined in claim 10, wherein the tray surface panel, the first side panel and the first flap are all formed from the same integral piece of material.

14. An endoscopy tray comprising:
a tray surface panel having an upstanding wall extending therefrom, the wall including a plurality of wall elements joined at their ends to form an enclosed perimeter, the wall elements and tray surface panel defining a fluid retaining space;
a first side panel extending from a first side edge of the tray surface panel and rotatable relative thereto between a storage position and an operative position, the first side panel having a first flap rotatably secured thereto, the flap having an engagement element selectively securable to a first wall element of the plurality of wall elements for securing the first side panel in an operative position, the first side panel having a distal end including a first recess adapted to retain a tube end above the fluid retaining space.

15. The tray as defined in claim 14, wherein the tray surface panel has a second side edge opposed from the first side edge and a second side panel extends from the second side edge of the tray surface panel and rotatable relative thereto between a storage position and an operative position, the second side panel having a flap rotatably secured thereto, the flap having an engagement element selectively securable to a second wall element of the plurality of wall elements for securing the second side panel in an operative position.

16. The tray as defined in claim 15, wherein the second side panel has a distal end including a first recess adapted to retain a tube end above the fluid retaining space.

17. The tray as defined in claim 16, wherein the tray surface panel has a tray retainer extending from a third side edge disposed between the first and second side edges.

18. The tray as defined in claim 15, wherein the first flap engagement element includes a notch formed therein, the notch being configured to receive therein a portion of the first wall element.

19. The tray as defined in claim 15, wherein the tray surface panel, the first side panel and the first flap are formed from one integral component.

20. The tray as defined in claim 14, wherein the first side panel has a second flap rotatably secured thereto, the second flap having a retaining element formed therein and being engagable with the first wall element.

21. A foldable endoscopy tray comprising:
a tray surface panel having a fluid retaining space defined between a first and side edge of the tray surface panel;
a first side panel extending outwardly from the first side edge of the tray surface panel and a second side panel extending from the second side edge of the tray surface panel, the first and second side panels each being rotatable relative to the tray surface panel between a storage position and an operative position;
the first side panel having a first flap rotatably secured thereto and the first flap having an engagement element, wherein the first flap is foldable relative to the first side panel into a position wherein the engagement element engages the tray surface panel to secure the first side panel in an operative position, and the first side panel having a distal end including a first recess adapted to retain a first tube end above the fluid retaining space; and
the tray surface panel has a third side edge disposed between the first and second side edges and a tray retainer extends from the third side edge.
